(19) United States
(12) Plant Patent Application Publication
(10) Pub. No.: US 2002/0095707 P1
(43) Pub. Date: Jul. 18, 2002

(54) Seashore paspalum 'SDX-1'

(76) Inventors: Michael DePew, Tekonsha, MI (US); Stewart Bennett, Bokeelia, FL (US); Paul H. Tillman, Bokeelia, FL (US)

Correspondence Address: Laura G. Barrow, Esq., P. O. Box 215, Estero, FL, 33928-0215, US

(21) Appl. No.: 09/759,481

(22) Filed: Jan. 12, 2001

(51) Int. Cl. A01H 5/00

(52) U.S. Cl. PLT/388

(57) ABSTRACT

A novel seashore paspalum cultivar (Paspalum vaginatum) is disclosed. The ‘SDX-1’ cultivar is characterized by having a fine texture, greater sod strength, increased rooting, improved color, improved density, and greater salinity tolerance. The cultivar is particularly well suited for golf courses, grass tennis lawns, lawn bowls, lawns, and athletic fields.

### SUMMARY OF THE INVENTION

The present invention relates to a new cultivar of seashore paspalum (Paspalum vaginatum Swartz) that is particularly suited as a turf grass for recreational fields and golf courses. The new cultivar of the present invention is herein referred to by its cultivar name ‘SDX-1. ’

The ‘SDX-1’ cultivar is a low growing halophytic grass spreading by rhizomes and stolons. The ‘SDX-1’ cultivar exhibits a dwarf growth habit that seldom exceeds two inches in mature height under conditions of full sun. The grass is particularly well suited for use on golf courses for putting greens mowed to ⅛ inch and for fairways mowed to ¼ inch. It is also suitable for lawns, grass tennis courses, lawn bowls, athletic fields or any other area where a fine textured close mown and low growing turf with superior salt tolerance is desired.

### BRIEF DESCRIPTION OF THE DRAWINGS

FIG. 1 is photograph of the ‘SDX-1’ variety (#1) seed head compared to other seed head varieties of seashore paspalum (#2=applicants' ‘SGX-6’; #3=applicants' ‘SFX-14’; #4=applicants' ‘Creeping Bahiagrass’; and #5=native seashore paspalum.

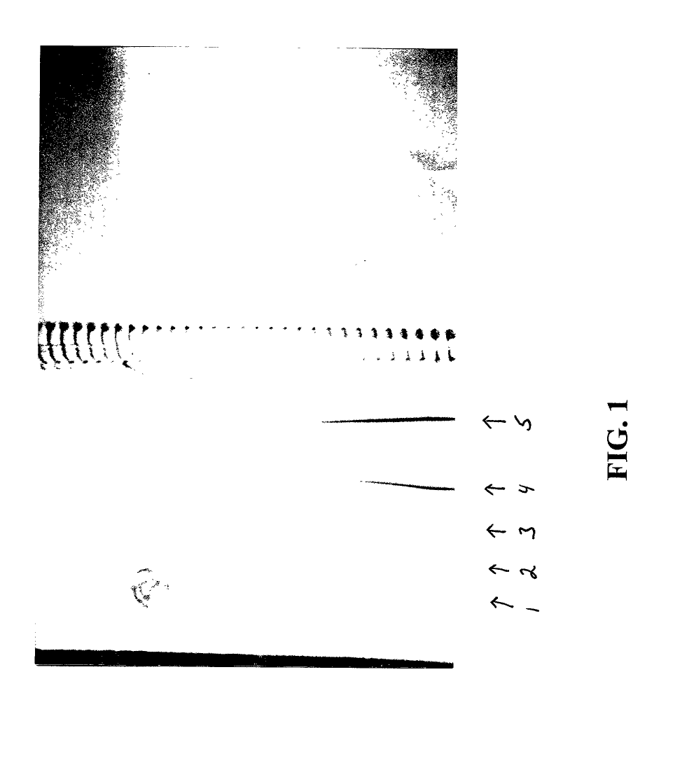

FIG. 2 is a photograph of core sod samples of the ‘SDX-1’ variety (#1) cut (unmown) compared to other core samples of seashore paspalum (#2=applicants' ‘SGX-6 (cut to 7/32 inch); #3=applicants' ‘SFX-14’ (cut to 9/16 inch); #4=‘Creeping Bahiagrass’ (cut at 3 or more inches).

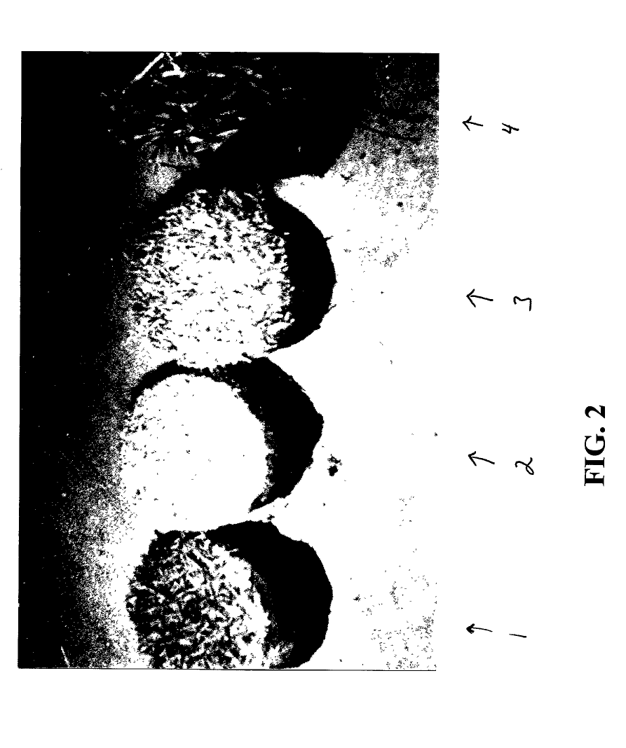

FIG. 3 is a photograph of stolon sprigs of the ‘SDX-1’ variety (#4) compared to other seashore paspalum varieties (#1=native seashore paspalum; #2=applicants' ‘SFX-14’ variety; and #3=applicants' ‘SGX-6’ variety).

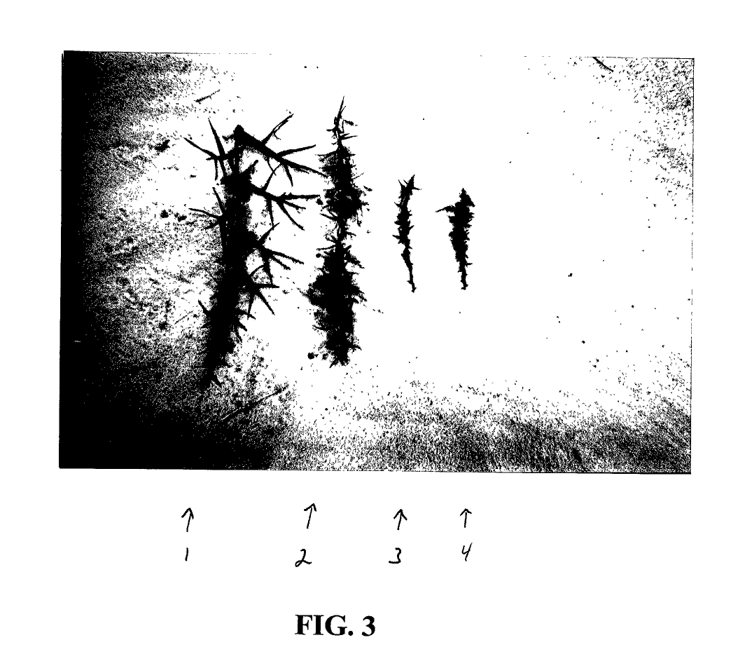

FIG. 4 is a photograph of the ‘SDX-1’ variety 4 weeks following sprigging.

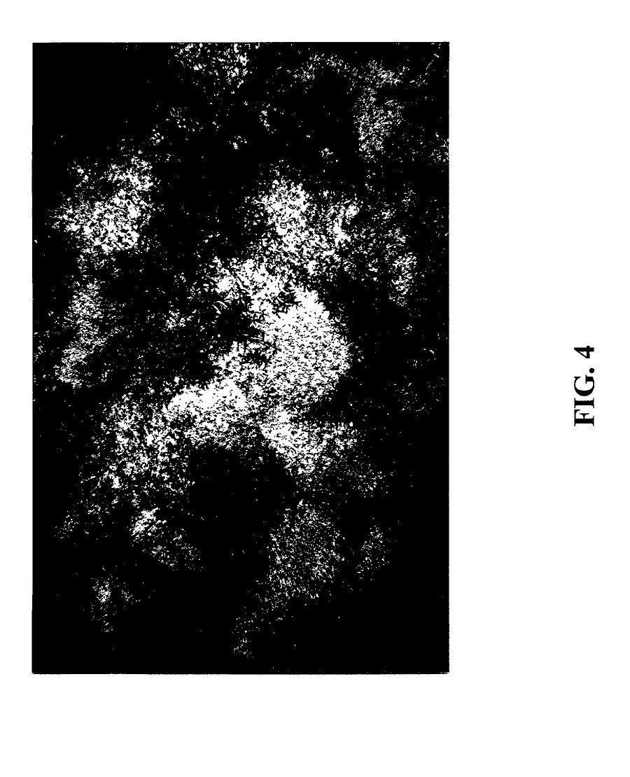

FIG. 5 is a photograph of the ‘SDX-1’ variety (unmown) 12 weeks following sprigging.

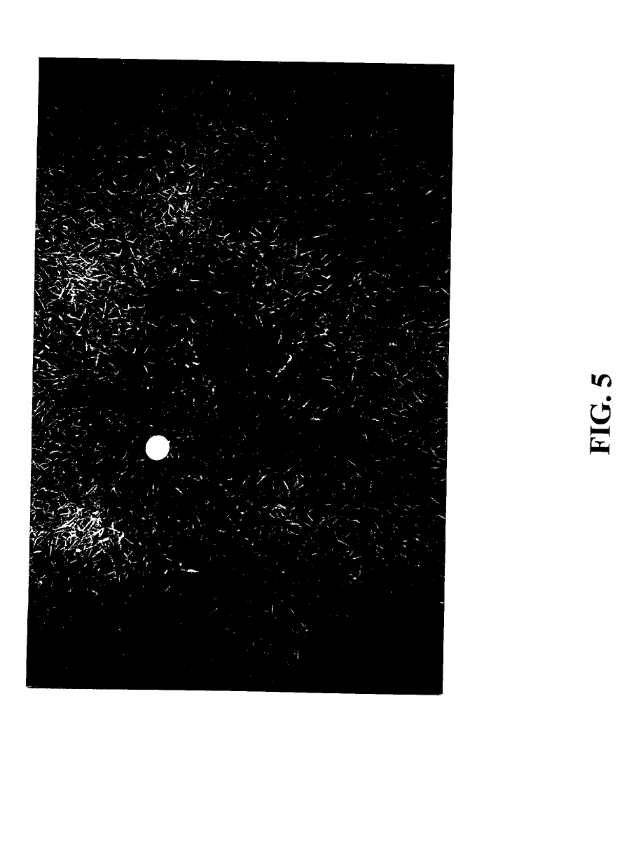

FIG. 6 is a photograph of the ‘SDX-1’ variety (unmown) 32 months following establishment.

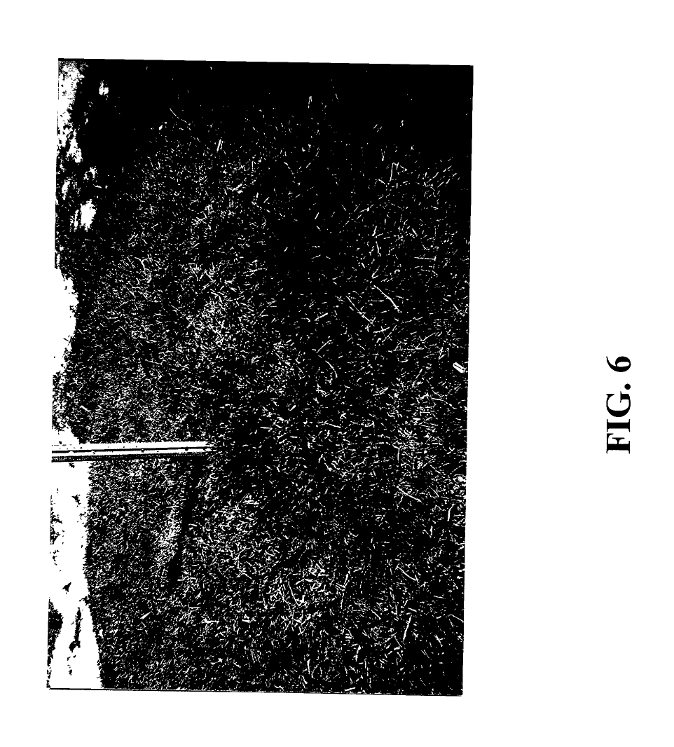

### DETAILED DESCRIPTION OF THE VARIETY

The following is a detailed description of the new grass variety based upon observation of the grass grown in field plots and under live golf conditions on a local golf course.

Asexual Reproduction:

The ‘SDX-1” grass was developed by the applicants on Pine Island, Fla. in a saline environment. Early ecotypes from the ETS turf development program were developed from crosses between Florida native ecotypes and ‘Adalayd’ seashore paspalum. ‘SDX-1’ was developed by crossing improved ETS turf-type ecotypes and subsequent evaluation of the resulting progeny. Trial areas of the grass were established and maintained under live golf conditions at Alden Pines Country Club, Bokeelia, Fla. (a salt water irrigated golf course) since 1997. This grass has been asexually propagated by the applicants as sprigs, plugs, and sod.

Botanical Description:

The ‘SDX-1’ cultivar is a perennial grass. The grass stems have overlapping sheath margins and arise from an extensive system of long, straw colored, slender rhizomes and/or purple-tinged stolons. The grass blades are glabrous and folded, forming a V-shape. The grass blades have an olive green color, Munsell color designation 7.5 GY 4/6 (abaxial side) and 7.5GY 5/4 (adaxial side). The blade length is generally 0.5-1.6 cm (unmown). The blades have a typical width of from 1.75 to 3.0 mm, varying with the level of salinity and tapering to an involute apex. Upon flowering, the culms are about 1.5-4.5 cm tall with inflorescence forming paired branches (racemes) at the culm apex. The racemes are about 0.6 to 1.6 cm in length, each having a broad, triangular rachis bearing two rows of spikelets (one floret per spikelet).

Leaf blade width and length of the ‘SDX-1’ cultivar are affected by both salinity level and by the length of sunlight exposure. It produces longer narrower leaf blades in winter compared to shorter, wider leaf blades in summer. Likewise, as salinity levels increase, shorter, narrower, and somewhat more erect leaf blades are produced, compared to longer, wider blades produced upon irrigation with a fresh water or low salinity system.

General Observations:

This grass has shown to be particularly well suited for use on putting green surfaces due to the high shoot density, short internode length, short blades and fine texture produced under close mown conditions and saline irrigation. Due to its inherent dwarf growth form, this grass is also suited for use on high-quality lawn areas where an infrequent mowing regime is utilized, seldom exceeding 2 inches in overall height under unmown conditions in full sunlight. While ‘SDX-1’ exhibits dwarf growth form with short internode length and overall low vertical growth habit, the grass grows very vigorously and densely in a horizontal fashion and quickly forms a dense sod when established from sprigs. This grass has been tested under regular saline irrigation up to 26,000 ppm. Trials have been conducted applying 45,000 ppm (brine) salinity water to putting green surfaces established with this grass for weed and pest control. Target organisms have been eliminated with the brine water application with no apparent visual damage to the grass. Direct applications of table salt or sea salt has also been employed for weed control although temporary burning of the grass leaf tips may be observed with heavy salt applications.

Compared to ‘Adalayd’ (‘Excalibre’) seashore paspalum utilized for similar purposes, this grass has foremost greater salinity tolerance, and under saline conditions, improved density, finer texture, greater sod strength, increased rooting and improved color. Compared to other Seashore paspalum turfgrass ecotypes developed in the Environmental Turf Solutions, Inc. (ETS) turf development program, ‘SDX-1’ is a compact, dwarf form that forms a low, dense, tight sod even under unmown conditions. Compared to other fine textured grass species (notably common and hybrid bermudagrass) for similar use, this grass has adaptations for moderate shade tolerance and enhanced temperature tolerance, retaining color at lower temperatures than bermudagrasses. This grass has a lower water use requirement compared to St. Augustine and hybrid bermudagrass cultivars and has enhanced qualities for drought tolerance and can be grown in sandy soils as well as in heavy-textured waterlogged soils.

We claim:

1. A new and distinct cultivar of seashore paspalum grass, substantially as herein illustrated and described, characterized by its fine texture when closely mown, its superior tolerance to salt, drought, and soil hypoxic conditions, and its low, dwarf growth habit even under unmown conditions.
